(19) United States
(12) Patent Application Publication
(10) Pub. No.: US 2014/0065183 A1
(43) Pub. Date: Mar. 6, 2014

(54) CONJUGATES OF ACYL HOMOSERINE LACTONE AND CATALASE A FROM PSEUDOMONAS AERUGINOSA

(71) Applicants: Jennelle M. Kyd, Rockhampton (AU); Margaret Cooley, Hobart (AU)

(72) Inventors: Jennelle M. Kyd, Rockhampton (AU); Margaret Cooley, Hobart (AU)

(73) Assignee: Central Queensland University, Rockhampton, Queensland (AU)

(21) Appl. No.: 13/997,409

(22) PCT Filed: Dec. 23, 2011
(86) PCT No.: PCT/AU11/01685

§ 371 Date: Sep. 9, 2013

(30) Foreign Application Priority Data
Dec. 24, 2010 (AU) 2010905632

(51) Int. Cl. A61K 39/385

(52) U.S. Cl. CPC A61K 39/385
USPC 424/197.11; 435/188

(57) ABSTRACT

The present invention is directed to an acyl homoserine lactone and catalase conjugate wherein the acyl homoserine lactone is N-3-(oxododecanoyl)-L-homoserine lactone (OdDHL) or butyryl L-homoserine lactone (BHL) and the catalase is a P. aeruginosa KatA protein or an antigenic portion thereof. The conjugate is used to treat P. aeruginosa infections by limiting the formation of biofilms and inhibiting a range of quorum-sensing dependent virulence factors by having an immunogenic conjugate provided as a therapeutic or prophylactic vaccine.

### FIELD OF INVENTION

The present invention relates to immunogenic conjugate molecules. The present invention relates to conjugates of molecules associated with quorum sensing involved in biofilm formation and more particularly acyl homoserine lactone conjugates.

### BACKGROUND OF THE INVENTION

Persistent infections resulting from Pseudomonas aeruginosa are the major cause of morbidity and mortality in cystic fibrosis (CF) patients, and in those with bums, neutropenia or with otherwise compromised immunity. P. aeruginosa is notoriously difficult to eradicate even with long-term antibiotic therapy and there is evidence that the ability of the organism to form biofilms contributes to this resistance.

Biofilm formation is controlled by small quorum sensing signal molecules (QSSMs), including a number of different acyl homoserine lactones (AHLs). The most frequently produced acyl homoserine lactones are N-3-(oxododecanoyl)-L-homoserine lactone (OdDHL) and butyryl L-homoserine lactone (BHL). However acyl homoserine lactones appear not to be immunogenic and have been shown to be weakly immunogenic when conjugated to a general carrier protein.

### OBJECT OF THE INVENTION

It is an object of the present invention to provide a conjugated AHL protein that is immunogenic that overcomes at least in part one or more of the above mentioned problems.

### SUMMARY OF THE INVENTION

The current invention was developed from the concept that blocking quorum sensing signal molecules could limit the ability of P. aeruginosa to form biofilms and inhibit a range of quorum-sensing dependent virulence factors. The approach to block QSSM was to develop an immunogenic AHL which can be used as a therapeutic or prophylactic vaccine. The approach was to conjugate AHL with the immunogenic P aeruginosa protein KatA. KatA is a P aeruginosa catalase protein that decomposes hydrogen peroxide released from phagocytes (Thomas L D, Dunkley M L, Moore R, Reynolds S, Bastin D A, Kyd J M, et al. Catalase immunization from Pseudomonas aeruginosa enhances bacterial clearance in the rat lung. Vaccine 2001; 19:348-57). The strategy was to develop an immune response to AHL to reduce the ability of P aeruginosa to form biofilms, while at the same time induce an immune response to the conjugated protein KatA, to neutralize the bacterial catalase functions and make P aeruginosa more susceptible to host immune defences.

In one aspect the present invention broadly resides in an acyl homoserine lactone and catalase conjugate.

Preferably the catalase is a Pseudomonas aeruginosa catalase or an antigenic portion thereof. More preferably the catalase is a P. aeruginosa KatA protein or an antigenic portion thereof.

The acyl homoserine lactone is any suitable acyl homoserine lactone molecule or antigenic portion thereof. Preferably the acyl homoserine lactone is an acyl homoserine lactone from P. aeruginosa. More preferably the acyl homoserine lactone is N-3-(oxododecanoyl)-L-homoserine lactone (OdDHL) or butyryl L-homoserine lactone (BHL).

In another aspect the invention broadly resides in an antigenic composition including an acyl homoserine lactone and catalase conjugate to induce an immune response. Preferably the immunogenic composition is a vaccine composition further including one or more suitable adjuvants. Suitable adjuvants include inorganic gels such as aluminum hydroxide or water oil emulsions such as incomplete Freund's adjuvant.

In a further aspect the present invention is a method of treatment of a P. aeruginosa infection in a patient with an antigenic composition including an acyl homoserine lactone and catalase conjugate to induce an immune response.

In another aspect the present invention is the use of an antigenic composition including an acyl homoserine lactone and catalase conjugate in the treatment of a patient with a P. aeruginosa infection.

The preferred features of each aspect of the invention also apply to each other aspect.

The invention will now be described with reference to the following preferred embodiment. The preferred embodiment is provided by way of example and the invention is not limited by or to the preferred embodiment.

### BRIEF DESCRIPTION OF THE DRAWINGS

In order that the present invention can be more readily understood reference will now be made to the accompanying drawings which illustrate a preferred embodiment of the invention and wherein:

FIG. 1 is an SDS-PAGE and coomassie brilliant blue staining of KatA expression and purified product wherein Lane M is a molecular mass ladder with band mass indicated on the left, lane 1 is non induced cells; lane 2 is induced cells showing KatA protein band; lane 3 is a cell lysate; lane 4 is a flow through fraction; lanes 5 & 6 are washes; lanes 7 & 8 are elution fractions containing purified KatA.

FIG. 2 is a Western Blot from SDS-PAGE of KatA expression and purified product wherein Lane M is a molecular mass ladder with band mass indicated on the left, lane 1 is non induced cells; land 2 is induced cells showing KatA protein band; lane 3 is a cell lysate; lane 4 is a flow through fraction; lanes 5 & 6 are washes; and lanes 7 & 8 are elution fractions containing purified KatA.

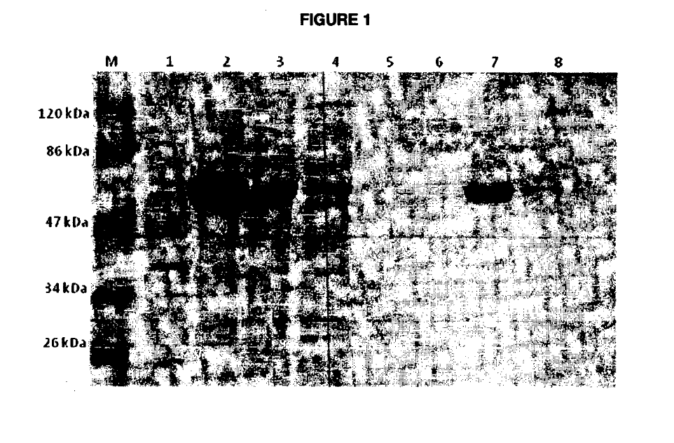

FIG. 1 is a coomassie stained SDS-PAGE of the two carrier proteins BSA and KatA, and their OdDHL conjugates wherein Lane M is the molecular mass ladder; Lane 1 is BSA prior to conjugation; Lane 2 is BSA-nhc (control); Lane 3 is BSA-OdDHL; Lane 4 is blank; Lane 5 is molecular mass ladder; Lane 6 is KatA prior to conjugation; Lane 7is KatA-nhc (control); and Lane 8 is KatA-OdDHL.

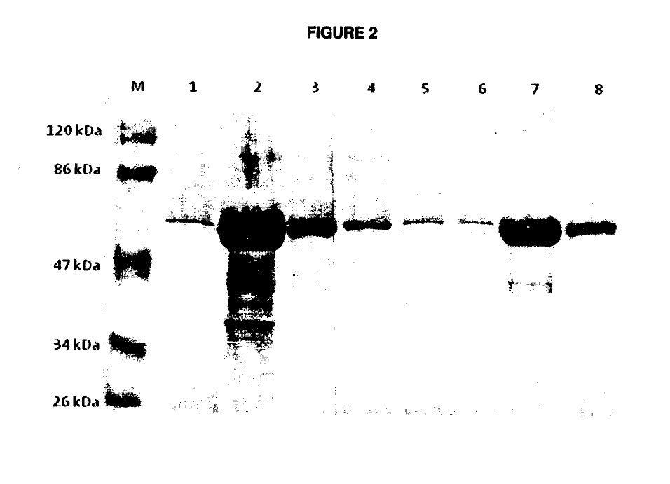

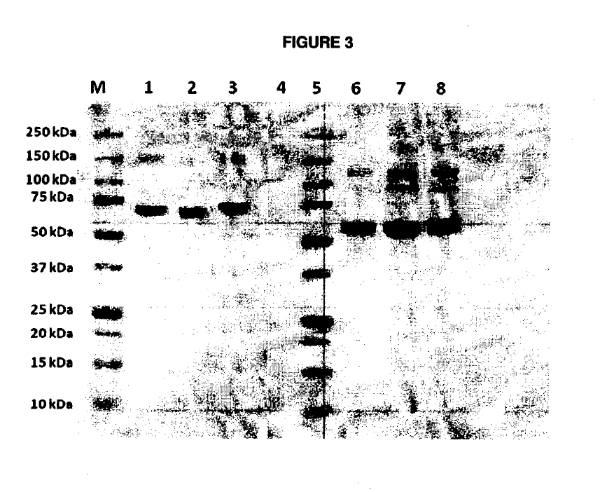

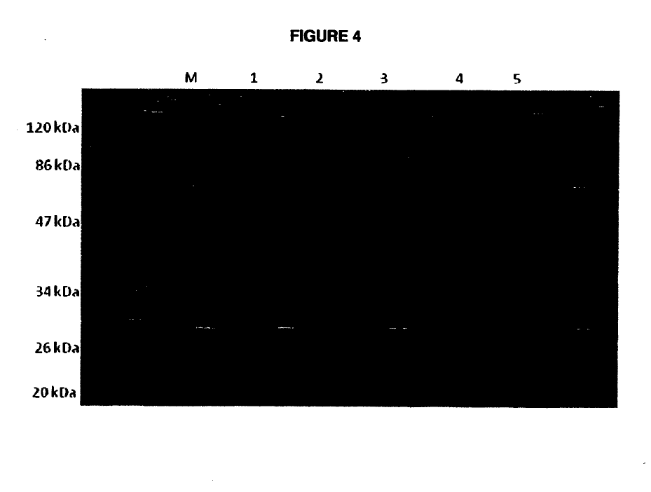

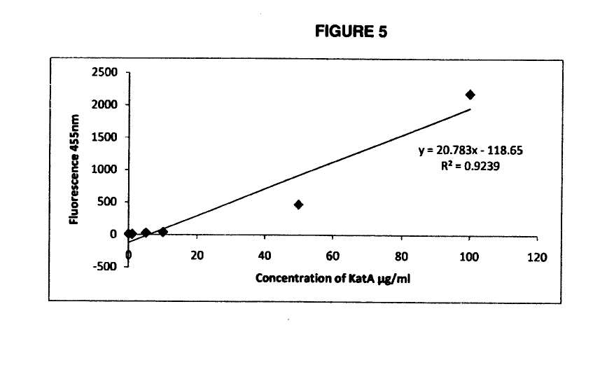

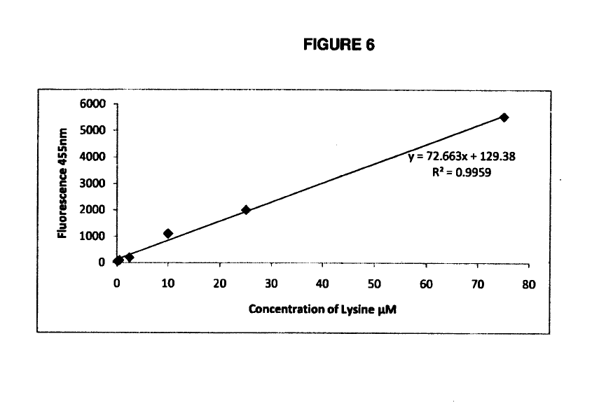

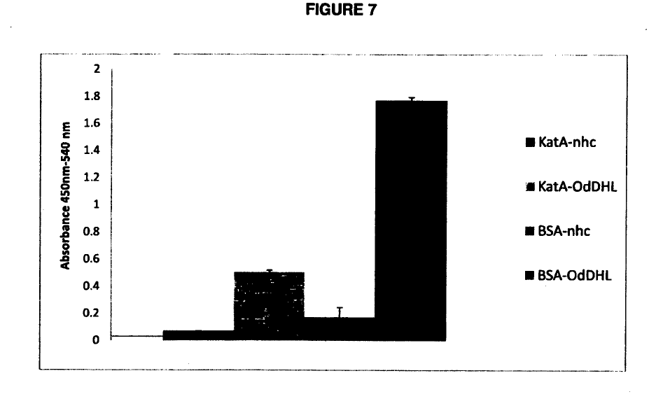

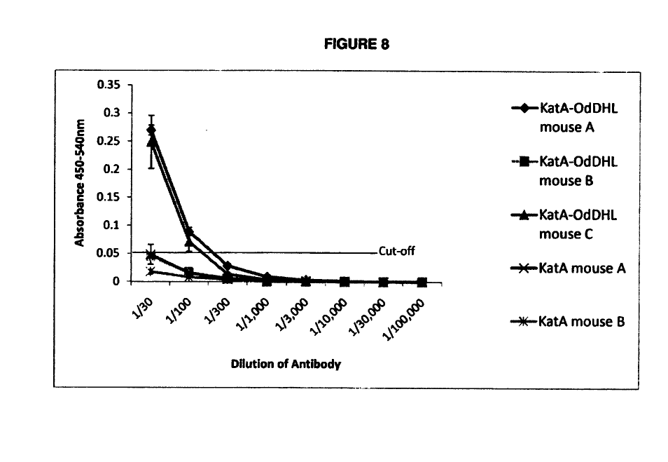

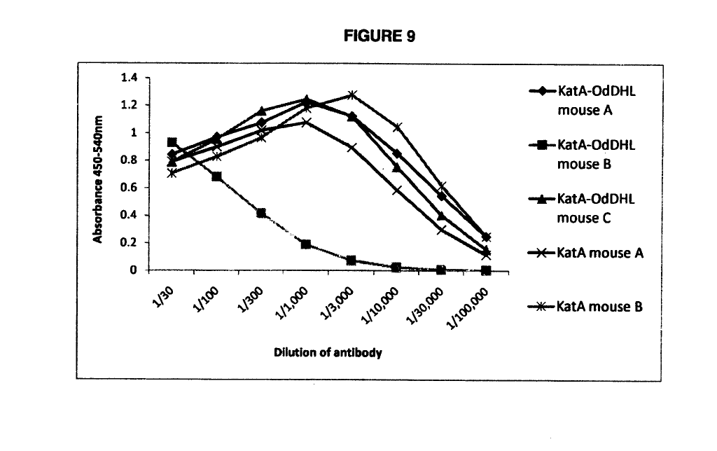

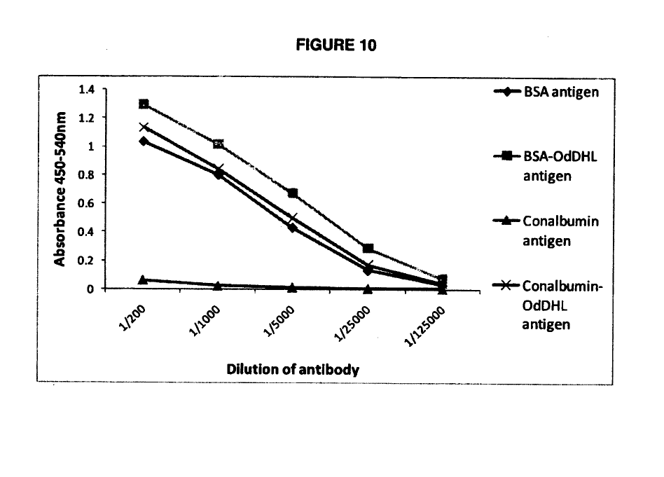

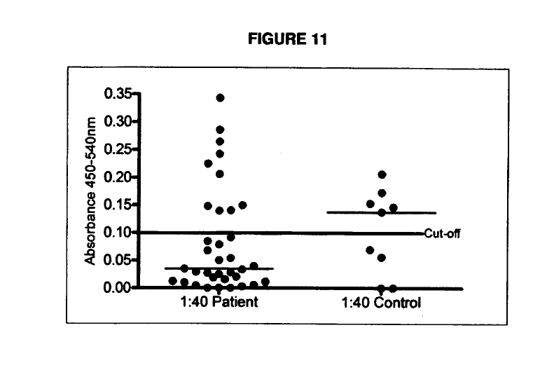

FIG. 2 is a Western blot using mouse anti-KatA antibody to detect purified KatA protein and KatA expressed in P. aeruginosa PA01 lysate, wherein Lane M is a molecular mass ladder; lane 1 is a pre-conjugated KatA; lane 2 is a KatA-no hapten control; lane 3 is a KatA-OdDHL; lane 4 is a KatA expressed in P. aeruginosa PA01 lysate; and lane 5 is a molecular mass ladder.

FIG. 5 is an OPA assay standard curve for KatA.

FIG. 6 is an OPA assay standard curve for Lysine.

FIG. 7 is a graph showing the detection of conjugated OdDHL on KatA using sera from Conalbumin-OdDHL immunised mice.

FIG. 8 is a graph of two of three KatA-OdDHL immunised mice that produce antibodies to OdDHL wherein BSA and BSA-OdDHL antigens at 5 μg/mL were coated onto Nunc Maxisorp plates and probed with dilutions of sera from mice immunised with KatA-OdDHL or KatA only.

FIG. 9 is a graph of antibodies to KatA in mice immunized with either KatA or KatA-OdDHL wherein KatA at 5 μg/mL was coated onto Maxisorp plates and probed with sera from KatA-OdDHL and KatA immunised mice.

FIG. 10 is a graph of both BSA and OdDHL antibodies generated in mice that were specific for their conjugate antigens, wherein all antigens were diluted to a concentration of 10 μg/mL, coated onto Nunc Maxisorp plates and probed with BSA-OdDHL sera and control sera.

FIG. 11 is a diagram showing individuals within both the CF patient group and the control group that have antibodies that are reactive to OdDHL wherein the BSA and BSA-OdDHL was at a concentration of 5 μg/mL and was applied to Nunc Maxisorp plates and probed with BSA-OdDHL sera and control sera.

### DETAILED DESCRIPTION OF THE PREFERRED EMBODIMENT

### Methods and Results

### Expression and Purification of KatA

The method of expression and purification involves using standard procedures known to the person skilled in the art and they can be easily altered to other suitable systems to express the KatA gene in a different expression vector and for scale-up processes suitable for production of vaccine-product quantities of the recombinant protein. The KatA gene was isolated from P. aeruginosa strain 385 (Thomas L D, Dunkley M L, Moore R, Reynolds S, Bastin D A, Kyd J M, et al. Catalase immunization from Pseudomonas aeruginosa enhances bacterial clearance in the rat lung. Vaccine 2001; 19:348-57) and has been accessed from P. aeruginosa strain PA01 (repository origin ATCC 47085).

### Chromosomal DNA Extraction.

A culture of Pseudomonas aeruginosa was washed in PBS and the pellet resuspended in 50 mM Tris-HCl, pH8.0. 0.4M EDTA was added and the mix incubated in a 37° C. water bath before adding 20 mg/ml lysozyme. Following incubation at 37° C., Proteinase K, 10% SDS and 10 mg/ml Ribonuclease A was added, incubated further at 37° C. until the suspension was clear. Phenol (saturated with 10 mM Tris-HCl and 1 mM EDTA) was added and gently mixed at 37° C. Following centrifugation at 8,000 rpm for 15 mins at 4° C., the DNA containing phase was removed and extracted in phenol/chloroform/isoamyl alcohol (25:24:1). After mixing on ice and centrifugation at 8,000 rpm, the DNA phase was removed, transferred to a tube containing 5 ml chloroform/isoamyl alcohol (24:1) and centrifuged at 8,000 rpm at 4° C. This step was repeated before the DNA was removed and 2 volumes of cold (−20° C.) absolute ethanol was used to precipitate the DNA. The DNA was extracted with a glass pipette, dipped in 70% ethanol and resuspended in TE (1 mM EDTA, 10 mM Tris-HCl, pH8).

### PCR

A standard procedure known to the person skilled in the art was employed and consisted of 0.2 μl 50 μM primer 1 and 0.2 μl 50 μM primer 2, 0.4 μl 10 mM dNTPmix, 10× reaction buffer (Fisher Biotech #201203), 0.2 μl Taq DNA polymerase, 1.3 μl 25 mM MgCl₂, 13.7 μl nanopure water. 2 μl template DNA (approx 5 ng/μl) was added. Sealed capillary or tube was placed into the thermocycler and the program started.

Sequence of Oligonucleotide Primers for KatA:

| | |
|---|---|
| 5′ | 5′CGGGATCCGAAGAGAAGACCCGCCTG 3′ forward |
| 3′ | 5′ GGGAAGCTTGCGTGTTGAGGTAATCGATGA 3′ |
| | reverse |

Underline denotes restriction sites

### DNA Digestion and Ligation

Standard procedures known to the person skilled in the art were used to digest and then mix insert DNA and plasmid vector. In separate tubes, insert DNA and plasmid DNA were digested with the digestion enzymes in the buffer recommended by the supplier for the enzymes. The digested DNAs were then mixed and incubated at 37° C. for 2 hours. Generally a ratio of 3:1 was used for insert:vector. Insert DNA and vector were mixed with 10× Ligation buffer (MBI Fermentas, Progen) and nanopure water (all kept on ice). Ligation enzyme (MBI Fermentas, Progen) was added and incubated at 16° C. overnight. Bacterial cells were made competent and in the production of KatA, was achieved by transforming the ligated plasmid into the competent Escherichia coli. Transformation was achieved using the CaCl₂ method, but can also be achieved by electroporation or heat shock.

### Induction and Extraction

Transformed bacteria were grown in LB broth containing the relevant antibiotic until they reached an optical density of between 0.7-0.9 at 600 nm. The protein expression was induced by addition isopropyl-β-D-thiogalactoside (IPTG) and the cells incubated for the relevant induction period. The bacteria were harvested by centrifugation at 4000×g, the pellet was resuspended in 50 mM sodium phosphate, 300 mM NaCl, pH8, 10 mM imidazole and frozen at −70° C. for between 0.5 h and overnight. The cell suspension was sonicated to disrupt the cells (but other disruption methods can be used). The suspension was centrifuged at 11,000 rpm to remove the debris and the supernatant retained for extraction of the recombinant protein using the Ni-NTA affinity resin (determined by the affinity tag expressed with the protein). The protein was extracted into phosphate buffer.

### Analysis of Purified KatA

SDS-PAGE was conducted to verify the production and purity of KatA (FIG. 1). A western blot was also used to detect the 6× His-tag co-expressed on the protein by probing with a mouse antibody specific to the His-tag and an anti-mouse-HRP secondary antibody (FIG. 2). The protein concentration was determined using a commercial assay (BCA assay samples were diluted in PBS or carbonate buffer (30 mM Na₂CO₃, 70 mM NaHCO₃).

### Conjugation of Activated Functionalised Carboxyl-OdDHL to Carrier Protein

An activated functionalised carboxyl-OdDHL was used as the conjugated hapten and the activated ester method was used to conjugate the activated functionalised carboxyl-OdDHL to the recombinant KatA protein [Hosoda H, Sakai Y, Yoshida H, Miyairi S, Ishii K, Nambara T. The preparation of steroid N-hydroxysuccinimide esters and their reactivities with bovine serum albumin. Chem Pharm Bull (Tokyo) 1979 March; 27(3):742-6.2]. The first step in the conjugation process was to activate the carboxyl-AHL. The hapten was dissolved in 100 μL of dimethyl formamide (DMF). Solutions of both N-hydroxysuccininimide (NHS) in DMF (100 mg/mL) and 1-ethyl-3-(3-dimethylaminopropyl)carbodiimide hydrochloride (EDC) in 100 mM carbonate buffer (100 mg/mL), pH 8.2 were added to the dissolved hapten and mixed well. The solution was incubated at 25° C. for 3 hours. KatA was diluted to a concentration of 1.89 mg/mL in 100 mM carbonate buffer, pH 8.2. The activated carboxyl-OdDHL was added to the protein solution and incubated at 25° C. for 2 hours. Purification of the conjugate was carried out against carbonate buffer by dialysis cassettes. After purification the conjugate was maintained by storing at −20° C. In addition, bovine serum albumin (BSA) was conjugated to activated carboxyl-OdDHL using the same procedure for the KatA conjugation.

### Characterisation of KatA-OdDHL Conjugate.

SDS-PAGE (FIG. 3) and western blot (FIG. 4) analyses were performed. The western blot was probed with mouse anti-KatA primary antibody produced by immunizing mice with KatA. Standard laboratory protocols were used. The efficiency of hapten-to-carrier protein conjugation was determined using a fluoraldehyde™ o-phthalaldehyde (OPA) assay (Table 1, FIGS. 5 and 6).

TABLE 1
Fluorescence values of the conjugate and the control.

| | 1/20 | 1/200 |
|---|---|---|
| KatA-OdDHL | 680.48 | 83.54 |
| KatA-NHC | 692.74 | 64.31 |

An ELISA was also conducted to determine whether the KatA did indeed have OdDHL conjugated to it (FIG. 7). The ELISA was able to confirm that KatA-OdDHL antigen coated plates showed a significantly greater signal with mouse anti-conalbumin-OdDHL sera than did KatA antigen alone (p=<0.001).

Nunc Maxisorp plates were coated with 100 μL of the diluted coating protein as indicated. The sealed plate was incubated overnight at 4° C. and then washed in 0.05% TWEEN in PBS (PBST). The plates were blocked with 2% skim milk power in PBST at room temperature for one hour, then washed. Dilutions sera in blocking buffer were added to wells and incubated overnight at 4° C. The plates were washed, then biotin labeled conjugated secondary antibody was added and incubated at room temperature for one hour. The plates were washed and Streptavidin-HRP was added. Following incubation at room temperature for one hour and further washing, the substrate was added and the plates were developed.

### Mouse and Inoculation Schedule

Male BALB/c mice were immunized with either the KatA-OdDHL conjugate or the KatA control protein. The mice were injected subcutaneously with 2.5 μL of protein (83 μg/kg body weight) in 25 μL PBS, mixed with 25 μL Freund's incomplete adjuvant (ICN Biomedicals, Aurora, USA). The schedule of injections is shown below

| Primary | 1^{st} | Preliminary | 2^{nd} | Terminal |
|---|---|---|---|---|
| Inoculation | Booster | Bleed | Booster | Bleed |
| Day 1 | Day 10 | Day 16 | Day 24 | Day 31 |

A preliminary tail bleed was performed at day 16 and a terminal bleed at day 31. After collection, blood was centrifuged at 10,000 rpm for two minutes to separate blood cells from the sera. The sera was collected and stored at −20° C.

Two of three mice showed significant antibody titres to OdDHL in their sera following immunization with the KatA-OdDHL conjugate (FIG. 8).

The immune responses of all mice toward the KatA antigen was very strong except in the mouse that had failed to also make antibodies to OdDHL (KatA-OdDHL mouse B). The response to KatA from all the other mice was exceeded an endpoint titre of 1/100,000 (FIG. 9).

The results showed that a conjugate vaccine that consists of KatA and OdDHL will elicit an immune response to OdDHL via the hapten-carrier effect. This supports that the vaccine construct will induce a strong antibody response following immunization.

### Human Immunity

Human blood was collected from a vein in the arm and stored in 4 mL EDTA anticoagulant tubes, fractionated by centrifuging it at 2000×g for 10 minutes at room temperature and the plasma layer aspirated with a 1 mL pipette. An equal volume of glycerol was added to each sample of plasma, mixed and the plasma stored at −80° C. until required.

Description of Patients and Controls

| | Patients | Controls |
|---|---|---|
| Number | 36 | 9 |
| Mean age (years) | | 32.26 |
| Age range (min, max) | | 22.92, 49.5 |
| Sex (M/F) | | |
| Documented pseudomonas | | 9/9 |
| infection (n/total n) | | |

The question was asked whether humans, particularly those with cystic fibrosis (CF) and infected with P. aeruginosa could naturally make antibodies to OdDHL. Plasma from 36 pediatric CF patients and nine healthy adult controls were tested in ELISA on plates coated with BSA or BSA-OdDHL antigens. For all sera, dilutions of 1/40, 1:400 and 1/4000 were tested. BSA was added to the human plasma before adding it to the plates to absorb any anti-BSA antibodies.

Ten of the 36 patients, (27. 8%) and 5 of 9 controls exhibited antibody reactivity to OdDHL (FIG. 11).

### Variations

It will of course be realised that while the foregoing has been given by way of illustrative example of this invention, all such and other modifications and variations thereto as would be apparent to persons skilled in the art are deemed to fall within the broad scope and ambit of this invention as is herein set forth.

Throughout the description and claims this specification the word “comprise” and variations of that word such as “comprises” and “comprising”, are not intended to exclude other additives, components, integers or steps.

1. An acyl homoserine lactone and catalase conjugate wherein the catalase is a Pseudomonas aeruginosa catalase or an antigenic portion thereof and the acyl homoserine lactone is any suitable acyl homoserine lactone molecule or antigenic portion thereof.

2. An acyl homoserine lactone and catalase conjugate as claimed in claim 1 wherein the catalase is a P. aeruginosa KatA protein or an antigenic portion thereof.

3. An acyl homoserine lactone and catalase conjugate as claimed in claim 1 wherein the acyl homoserine lactone is an acyl homoserine lactone from P. aeruginosa.

4. An acyl homoserine lactone and catalase conjugate as claimed in claim 1 wherein the acyl homoserine lactone is N-3-(oxododecanoyl)-L-homoserine lactone (OdDHL) or butyryl L-homoserine lactone (BHL).

5. An antigenic composition including an acyl homoserine lactone and catalase conjugate to induce an immune response wherein the antigenic composition includes one or more suitable adjuvants including inorganic gels such as aluminum hydroxide or water oil emulsions such as incomplete Freund's adjuvant.

6. An antigenic composition as claimed in claim 5 wherein the acyl homoserine lactone is N-3-(oxododecanoyl)-L-homoserine lactone (OdDHL) or butyryl L-homoserine lactone (BHL) and the catalase is a P. aeruginosa KatA protein or an antigenic portion thereof.

7. A method of treatment of a P. aeruginosa infection in a patient with an antigenic composition including an acyl homoserine lactone and catalase conjugate to induce an immune response.

8. A method of treatment as claimed in claim 7 wherein the acyl homoserine lactone is N-3-(oxododecanoyl)-L-homoserine lactone (OdDHL) or butyryl L-homoserine lactone (BHL) and the catalase is a P. aeruginosa KatA protein or an antigenic portion thereof.

9. The use of an antigenic composition including an acyl homoserine lactone and catalase conjugate in the treatment of a patient with a P. aeruginosa infection.

10. The use of an antigenic composition as claimed in claim 9 wherein the acyl homoserine lactone is N-3-(oxododecanoyl)-L-homoserine lactone (OdDHL) or butyryl L-homoserine lactone (BHL) and the catalase is a P. aeruginosa KatA protein or an antigenic portion thereof.
